(19) United States
(12) Patent Application Publication
(10) Pub. No.: US 2007/0262165 A1
(43) Pub. Date: Nov. 15, 2007

(54) System and method for altering the aroma within the head space of a container

(76) Inventor: Steven M. Landau, Meadowbrook, PA (US)

Correspondence Address: LAMORTE & ASSOCIATES P.C., P.O. BOX 434, YARDLEY, PA, 19067, US

(21) Appl. No.: 11/430,097

(22) Filed: May 9, 2006

(51) Int. Cl. A24F 25/00; A62C 13/62; A61L 9/04

(52) U.S. Cl. 239/34; 239/302; 239/51.5

(57) ABSTRACT

A packaging system for a consumable product held within a confined package. Plastic material is introduced within the confined packaging, preferably as a closure, seal or lining to the closed packaging. The plastic material has a surface that faces toward the consumable product within its packaging. The plastic material is fabricated from a composition that includes a plastic material and at least one scented material dissolved within that plastic material in a concentration in excess of its solubility at ambient temperature. The scented materials contained within the saturated plastic separates out of solution and migrates to the exposed surfaces of the plastic seal. The aroma volatiles are released from the plastic and fill the headspace of the packaging. These volatiles change the aromatic properties of the gases in the head space of the packaging.

### BACKGROUND OF THE INVENTION

1. Field of the Invention

In general, the present invention relates to the systems and methods of making scented plastics. The present invention also pertains to the use of scented plastics within containers that hold consumable products.

2. Prior Art Statement

Leaching is the process when oils or other compounds from a first material dissolve into a second material that contacts the first material. The adding of flavoring to consumable products through leaching has been in existence for thousands of years. Many wines are aged in barrels of different woods, wherein flavoring from the wood leaches into the wine and imparts a subtle flavor to the wine. Many fine liquors, such as scotch, brandy, whisky and the like also receive flavoring from the barrels in which they are stored and aged.

However, not all flavoring due to leaching is good. In modern manufacturing, many food and drink containers are made of molded plastic. Such containers include soda bottles, water bottles, milk bottles, juice containers, chewing tobacco containers and the like. When plastic is first molded, it goes through a period of degassing, where the molded plastic emits a strong scent. It is the degassing of plastic that is partially responsible for giving a newly manufactured car its “new car” smell. The period of degassing can last from several minutes to several years, depending upon the type of plastic being molded and the mass of the plastic present. Furthermore, as plastic ages and is exposed to light, air, moisture and environmental pollutants, the plastic degrades. As plastic degrades, the plastic emits oils, gas and other compounds.

When a liquid or moist solid is stored in a plastic container, the emissions from the plastic caused by degassing and degradation are dissolved and/or mixed with the contents of the container. The emissions from the plastic have an adverse affect on the flavor of the container's contents, often causing liquids and solid consumables to taste “like plastic” or “stale”. If the contents of a container are flavored and the contents are stored in a cold environment for a relatively short period of time, such as with soda in a soda bottle, then emissions from the plastic are usually not noticeable by a person consuming the stored product. However, if the stored product is not flavored and is not refrigerated, the adverse effects of the plastic leaching become more noticeable. For example, if plastic bottle is used to hold water and is carried by a hiker on a hot day, there is a good chance that the flavor of the water will be adversely affected by plastic leaching when the water is drunk.

When plastic leaches, the unfavorable compounds emitted by the plastic tend to collect in the head space of the container. The head space of the container is the pocket of air at the top of the container that exists between the opening the container and the top level of the consumable product held within the container. In a beverage bottle, the head space is the pocket of air that exists between the cap of the bottle and the top of the beverage.

When a container is opened by a consumer, the aroma of the air held within the head space is typically the first thing that can be detected by the consumer. If the head space air contains unpleasant plastic emissions the consumer's first impression of a product is that it smells “like plastic”. Likewise, if the head space contains an oxidized or degraded aroma of the food and beverages, a consumer's first impression may be that the product is old or has gone bad.

In the prior art, attempts have been made to use plastic leaching to the benefit of a product, rather than to the detriment of the product. Container closures have been made from plastic that is mixed with scented materials. The plastic therefore emits a pleasant aroma. If such plastic is used in a container, the foul emissions of the plastic are masked and the head space in the container tends to smell like the scent added to the plastic. Such a system is exemplified in U.S. Patent Application Publication No. 2002/0139093, to Landau, the applicant herein, entitled System And Method For Passively Adding Scent To A Consumable Product Using Plastic Leaching.

A problem associated with prior art systems that use traditional scented plastic, is that the container itself must contain large areas of plastic in order for the scented plastic to have a significant positive effect. The surface area of the plastic is more important than the mass of the plastic in such prior art systems. Since scented material is only mixed with the plastic, only the scented material at the surface of the plastic can ever dissociate from the plastic and enter the head space of the container. Thus, the technology is limited to all plastic containers or containers having large plastic closures or the container itself. Furthermore, making scented plastic by simply mixing scented material with plastic tends to perform best with low density plastic compositions, which could exclude a number of other plastic materials, as they do not perform as well. Low density plastics are pitted and contain many voids on a microscopic level. This increases the surface area of the plastic and provides the plastic with space to hold molecules of the scented materials. High density plastics do not have such voids and cannot retain any significant amounts of scented materials.

The packaging of many consumable products do not include low density plastics that can be scented in a traditional manner. Many beverages, for example, are bottled in glass containers and have metal closures. Such manufacturing techniques are typically used for bottles of beer. When a metal cap is used to cover a glass container, a very thin coating of a high-density plastic is typically applied to the underside of the cap. This thin seal is typically made of a poly-vinyl chloride (PVC) or a similar high-density thermoset plastic. The seal is very thin, typically only a few hundreds of an inch thick. The seal is also has a very small area, and is just large enough to cover the opening of the container. With such a volume of plastic and given the type of plastic, merely mixing scented materials with the plastic would produce a seal that emitted such a small amount of scent that it would have little or no effect upon the beverage being bottled. The heat of processing these materials is also unfriendly to the process, often resulting in volatilization of the preferred aromas. The traditional scenting of such metal-to-glass seals is not currently used in industry.

A problem therefore exists of how to effect the scent of the head space of a glass container having a metal cap. Manufactures want to effect this head space for many products. For example, light beer does not have the full-bodied aroma of traditional beer. Thus, when a consumer of a light beer opens a bottle or a can, they are not greeted with the same rich aroma as is presented by a bottle or can of regular beer. If the scent of the head space in a bottle of light beer can be effected, the scent of a full body beer can be added and the bottle of light beer would smell and taste more like a regular full bodied beer when opened. Since shelf life is often determined by aroma and taste, this would also potentially add the benefit of longer shelf life. Additionally since many beers are now being flavored, this technology offers the benefit of adding light fruity aroma and flavor to the beer.

A need therefore exists for a system and method of adding scent to the head space of a product whose packaging only contains a small volume of high density plastic. This need is met by the present invention as described and claimed below.

### SUMMARY OF THE INVENTION

The present invention is a packaging system for a consumable product having a container and a closure system for selectively closing and/or sealing the container. The packaging system is preferably configured as a cap structure that selectively engages the container thereby obstructing an access opening formed in the container. The cap structure has an interior surface that faces into the access opening toward the consumable product being packaged.

A plastic seal is attached to the interior surface of the cap or the bottle. The plastic seal is fabricated from a composition that includes a plastic material and at least one scented material dissolved or incorporated within that plastic material in a concentration in excess of its solubility at ambient temperature. The result is a plastic composition that is not at equilibrium. The scented materials contained within the supersaturated plastic separates out of solution and migrates to the exposed surfaces of the plastic seal. Additionally the aroma volatiles are released from the plastic and fill the headspace of the container or package component. Since the exposed surfaces of the plastic seal face the head space within the container, the scented material migrates volatiles into this space. These volatiles change the aromatic properties of the gases in the head space of the packaging, thereby altering the aroma perceived when the packaging is opened. Since equilibrium is achieved within the environment of the package, the flavor and aroma of the contents stay more consistent over time.

The flavor is not just randomly added to different plastic structures. Rather, the flavor types and concentrations are formulated in such a manner as to consider the heat history of the manufacturing process, compatibility with the material, the intended effect, and how the aroma dissipates from the material ultimately effecting the aroma of the product. Therefore, the additive must be carefully formulated in order to give the product a balanced profile which compliments the product rather than detracting from it.

### BRIEF DESCRIPTION OF THE DRAWINGS

For a better understanding of the present invention, reference is made to the following description of exemplary embodiments thereof, considered in conjunction with the accompanying drawings, in which:

FIG. 1 is a block diagram schematic that sets forth the methodology of producing a cap seal in accordance with the present invention;

FIG. 2 is a fragmented cross-sectional view of a first container system in accordance with the present invention;

FIG. 3 is a cross-sectional view of a second container system in accordance with the present invention;

FIG. 4 is a fragmented cross-sectional view of a third container system in accordance with the present invention;

FIG. 5 is a fragmented cross-sectional view of a fourth container system in accordance with the present invention, and

FIG. 6 is a cross-sectional view of a fifth container system in accordance with the present invention.

### DETAILED DESCRIPTION OF THE DRAWINGS

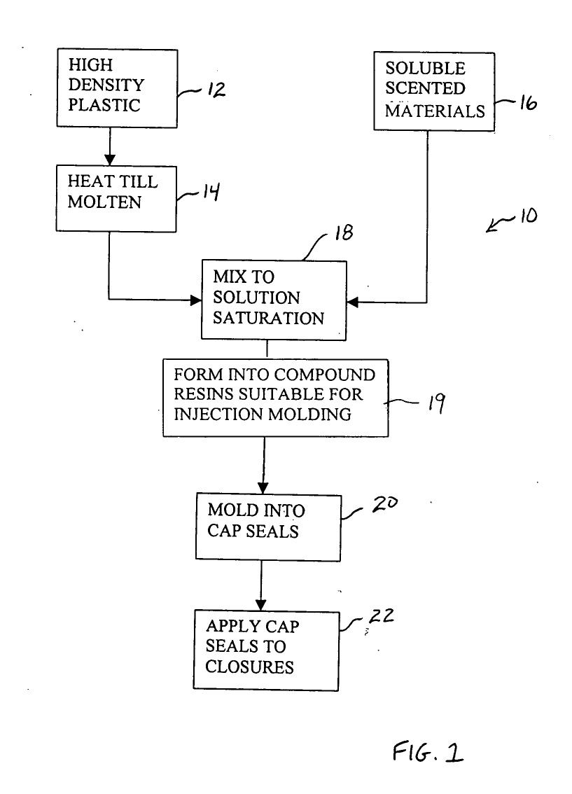

Referring to FIG. 1, the process 10 of molding a container seal in accordance with the present invention is shown. As is indicated by Block 12, traditional high density plastic is used in the formation of the container seal. The high density plastic can include PVC, PE, Plastisol or another such high density thermoset plastic polymer. The high density plastic is typically purchased in the form of bulk pellets, which may be custom blended and ready for molding.

The pellets of high density plastic polymers are filled into the supply hopper of a plastics forming machine. Within the machine is a heated chamber. The pellets of high density plastic are fed into the heated chamber where they are melted to a molten state of a precise temperature needed for molding. See Block 14.

As is indicated by Block 16, a supply of (flavor) scented material is provided. The scented material can be a single oil, a combination of oils or other aromatic materials which are capable of being soluble in plastic. The scented material selected has the aromatic characteristics desired by the manufacturer. The scented material selected is capable of being fully soluble in the plastic. The scented material is mixed with the molten plastic until the scented material is fully dissolved and integrated into the mix. The scented material is added to the plastic at a concentration that saturates the molten material. Thus, the scented material is dissolved within the molten material to such concentration as to create the best possible effect. See Block 18.

The molten plastic/scented material composition is cooled and turned into compounded resins (masterbatch concentrates). See Block 19. The compounded resins are used in the manufacturing of caps and container seals using traditional techniques. See Block 20. The container seals can be bottle caps, tamper seals, protective linings or the like structures typically used in the packaging of consumables. The seals are then applied to closures or containers in the traditional manner. See Block 22.

In the exemplary embodiment being illustrated, the molten plastic/scented material composition is cooled and turned into compounded resins which are then used in a molding process. However it is important to note that process is merely exemplary. Plastic/scented material can be added in other manners. For example, the plastic/scented material can be added to plastic as a direct liquid feed or as dry scented materials.

It will be understood that other additives may be added to the plastic in addition to the scented material. For instance, nanoclay particles can be added to the plastic. The use of such additives effects the amount of scented material that is to be added. For example, if nanoclay particles are added to the plastic, the presence of the nanoclay particles blocks the internal migration of the scented material. This creates a tortuous path for the scented material, thereby making the scented material migrate slower than it would normally. This prolonged migration may be desirable. If not desirable more scented material maybe added to compensate for the slowed release rate.

Once the seal is manufactured, it is cooled to a temperature below its molten point. The seal is therefore solid at the end of the process. The seal is then allowed to cool at ambient temperature, which may be over 200 degrees below its molten manufacturing temperature.

As the seals cool, the saturation point of the high density plastic falls in direct proportion to the decreasing temperature. The scented material that was dissolved within the high density plastic when it was molten is now in a concentration in excess of what can be absorbed by the high density plastic. Consequently, as the closure seal cools, the scented material supersaturates the high density plastic and separates out of solution. However, the closure seal solidifies before the scented material (flavor) has the opportunity to separate out of solution. The result is a plastic formulation that is out of equilibrium at ambient temperature. The plastic composition, however, tries to reach equilibrium. The scented material, therefore, slowly separates out of solution and migrates to the outer surfaces of the closure seal. The migration will continue until the scented material within the seal reaches equilibrium of solution at the ambient temperature of the seal. Although, the seal may be configured to be very thin, the migration is slow and will last for an extended period of time.

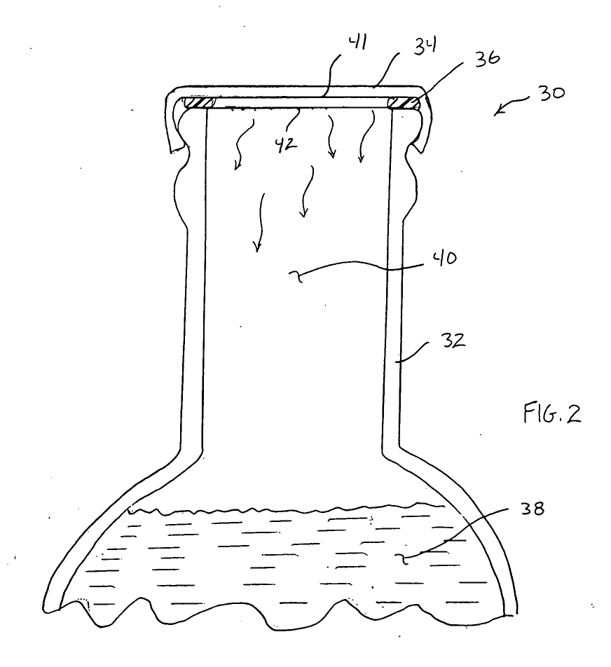

Referring to FIG. 2, a container assembly 30 is shown. The container assembly 30 includes a glass bottle 32 and a metal cap 34. The metal cap 34 has a cap seal 36 (also known as a gasket seal) present on its interior that is manufactured using the process described with reference to FIG. 1.

In FIG. 2, it can be seen that the bottle 32 is filled with a beverage 38 and that a head space 40 of gas exists below the cap seal 36 and above the beverage 38 within the bottle 32. The cap seal 36 creates an air impervious seal between the metal cap 34 and the rim of the bottle 32. The cap seal 36 has two flat surfaces 41, 42. The first flat surface 41 abuts against the interior of the metal cap 34. The first flat surface 41 is therefore isolated. As a consequence, scented material volatiles cannot separate out of solution along the first surface 41.

The second flat surface 42 of the cap seal 36 faces the head space 40 within the bottle 32. The second flat surface 42 is therefore not isolated. Most all of the scented material (flavor) volatiles that separate out of solution within the structure of the cap seal 36, will therefore migrate toward the second flat surface 42 and will become exposed to the head space 40. As a molecule of the scented material (flavor) becomes exposed to the head space 40, that molecule of scented material (flavor) can dissociate from the cap seal 36 and scent the head space 40 itself. The dissociation of the scented material molecules from the second flat surface 42 can be assisted by the beverage 38 within the bottle 32. As the bottle 32 is agitated during shipping and handling, the beverage 38 will splash against the cap seal 36 and wash the scented material molecules free.

Additionally aroma will also be released into the head space of the product through diffusion of volatiles into the air. Since the aroma is coming from the encapsulated aromatic plastic, the aroma profile is more stable than that which can be delivered directly from aromatics which are placed directly into the package contents. Consequently, the plastic encapsulation actually works both as a method to protect the aroma and to keep it stable, while also pushing the aroma out of the plastic.

Preferably, the layer with aromatic additive is directly positioned next to a layer which is an oxygen barrier. By positioning the aromatic layer next a barrier layer, which is often the outer layer of the package, the aroma is forced to migrate towards the contents inside the container.

As time passes, more and more scented material molecules and volatiles are released into the head space 40 of the bottle 32. After only a short period of time, the released scented material molecules and volatiles alter the scent of the gases present within the head space 40. As a result, then the metal cap 34 is opened and the gases of the head space 40 are released, that gas will include the distinct scent of the scented material volatiles that was added to the plastic composition of the cap seal 36. The concentration of the scent contained within the gases of the head space 40 increases over time until it reaches an equilibrium which is then maintained at a consistent level over time. Thus, instead of a beverage smelling more and more stale over time, the favorable aroma perceived for a beverage will actually stay stable for an increased period of time as volatiles, which are traditionally lost, are reintroduced back into the container from the packaging materials.

The scented material used in the plastic composition of the cap seal 36 is selected to complement the beverage 38 contained within the bottle 32. For instance, if the beverage 38 were a beer, the scented material may include the scent of fresh hops and/or barley to increase the fullness of the beverages aroma that is perceived when the bottle 32 is first opened. Similarly, if the beverage 38 were a soda, the scented material may include a complementary flavor, such as cherry, lemon, or vanilla that makes the olfactory bouquet of the beverage 38 more interesting and complex. Since the aromatic additive is protected by the plastic, it is much less prone to the oxidation and degradation effects that are typical of ingredients, thereby offering a fresher aroma profile.

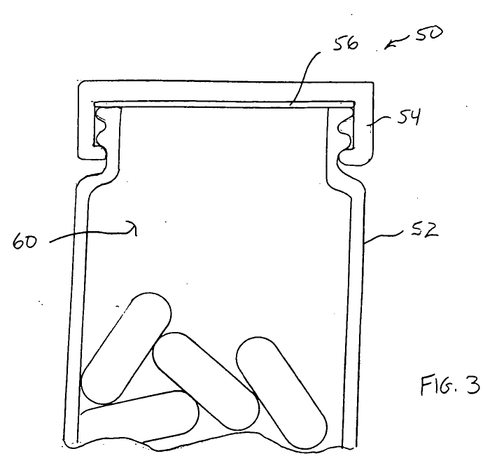

Referring to FIG. 3, an alternate embodiment of the present invention system is shown. In this embodiment, a pharmaceutical or nutraceutical container 50 is provided. The pharmaceutical/nutraceutical container 50 utilizes a plastic bottle 52 with a plastic cap 54. The plastic cap 54 includes an internal plastic seal 56 that seats against the rim of the plastic bottle 52. This could be achieved with either an induction seal or with a cap liner. Such seals are often used on pharmaceutical/nutraceutical containers that hold pharmaceuticals that are sensitive to humidity.

Pharmaceuticals and nutraceuticals are used for their medicinal properties. Many of the ingredients in these products often result in giving the product a bad aroma. Although pharmaceutical companies have worked diligently to try to enhance the aroma of their products and to mask malodors, they have been mostly unsuccessful since the ingredients they use often create an unfriendly environment for flavor and aroma additives. Pharmaceuticals and nutraceuticals therefore often produce head space gases that are very unpleasant to smell. By using a cap seal 56 of the present invention, molecules of scented material can be released into the head space 60 that offset the natural aroma of the pharmaceutical/nutraceutical. As result, the pharmaceutical/nutraceutical container 50 will release a neutral or pleasant aroma when opened, thereby making the taking of the pharmaceutical/nutraceutical much more palatable. Since the aroma is protected by being encapsulated within the plastic, it is not degraded or attacked by the other ingredients.

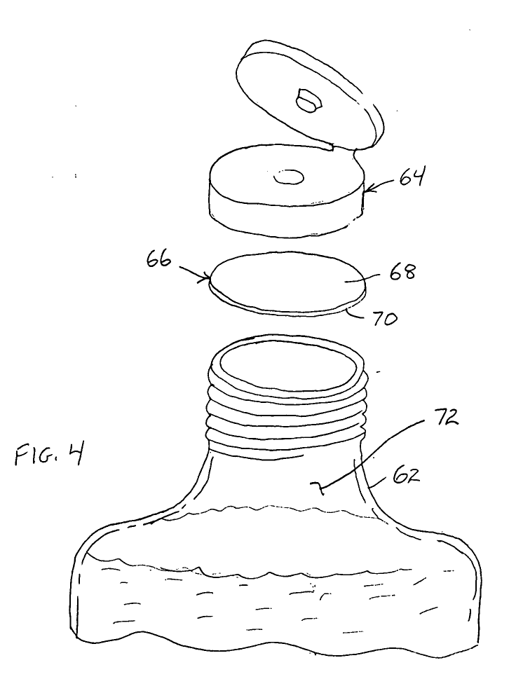

Referring to FIG. 4, another alternate embodiment of the present invention system is shown. In this embodiment, a beverage container 62 is shown having a sip cap closure 64. As is typically with such packaging configurations, a tamper seal 66 is placed over the opening of the beverage container 62. The tamper seal 66 is either a foil or paper substrate 68 coated with plastic material 70. The plastic material 70 is made from the same or similar plastic composition that has previously been described. As a result, the plastic material 70 will release molecules of scented material into the head space 72 of the beverage container 62 over time.

Since the plastic material 70 is just a thin film on the tamper seal 66, it is preferable that the substrate 68 be air impervious. This will cause the scented material in the plastic to migrate only in the direction of the head space 72 of the beverage container 62. Since the shelf life of beverage containers using such tamper seals is rather short, the small amount of plastic is sufficient to enhance the aroma of the head space 72 until the beverage container 62 is purchased and the beverage consumed.

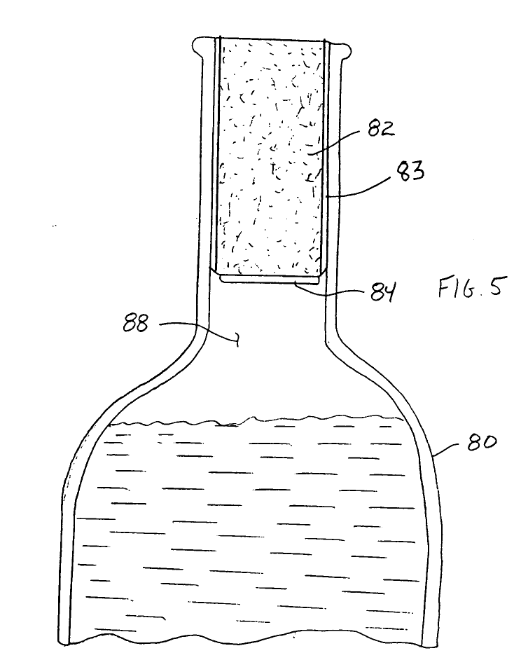

Referring to FIG. 5, yet another alternate embodiment of the present invention system is shown. In this embodiment, the container is a wine bottle 80 that is sealed with a cork 82. A synthetic cork infused with scented plastic of the type described here can be used. However, in the shown embodiment, a cork with a thin plastic seal 84 or skin, is being used. Alternatively, a cork with a plastic end cap, covering, medallion or outer skin material may be used. The seal 84 is applied or otherwise manufactured to the bottom of the cork 82 that faces the head space 88 within the wine bottle 80. The plastic seal 83 or skin 84 can be made from the same or similar plastic composition that has previously been described. As a result, the plastic seal layers 83, 84 will release molecules of scented material into the core of the cork and the head space of the bottle 88 over time. Since wine in a bottle 80 can be held for years, and since the aroma or bouquet of a bottle of wine is highly valued, the present invention can have a significant effect on the way a particular wine is perceived when opened.

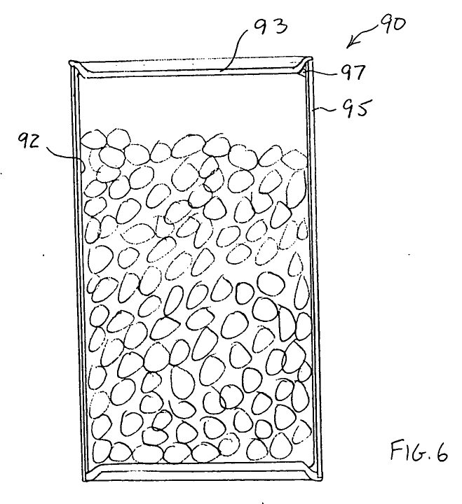

Referring to FIG. 6, another alternate embodiment of the present invention system is shown. In this embodiment, a can 90 is shown. The can 90 can be a metal can, such as is used with canned tomatoes, or an aluminum can, such as those used to hold beer. The can 90 is lined with a protective internal coating 92. The protective internal coating 92 contains the same or similar plastic composition that has previously been described. As a result, the protective internal coating 92 will release molecules of scented material into the headspace and contents of the can 90 over time. Depending upon the type of can being used, the can ends 93 can be joined to the cans body 95 with a plastisol material 97. The plastisol material is mixed with scented materials in the manner previously described to achieve the desired affect.

Although the protective internal coating 92 is just a thin film, the protective internal coating 92 covers the entire interior or the can 90. Consequently, the area of plastic material exposed to the interior of the can 90 is quite substantial. Furthermore, the metal of the can 90 is air impervious. This causes the scented material in the protective internal coating 92 or the plastisol 97 to migrate only in the direction of the interior of the can 90. This delivers a stable aroma and flavor profile to the contents of the can 90.

The illustrated containers and seals are merely exemplary of the many types of seals that are used to hold consumable products. In other forms, the seals can be applied to alternate containers such as jars, boxes or bags. Regardless to the structure of the container and its seal, it will be understood that the head space within the container is exposed to a volume of plastic that contains scented material in excess of its solubility. In this manner, molecules of the scented material will separate out of solution and it volatiles will migrate into the head space over time. It will therefore be understood that the embodiments of the present invention described and illustrated herein are merely exemplary and a person skilled in the art can make many variations to the embodiments shown without departing from the scope of the present invention. All such variations, modifications and alternate embodiments are intended to be included within the scope of the present invention as defined by the appended claims.

What is claimed is:

1. In a packaging system for a consumable product having a container with an access opening, a closure system for selectively closing said access opening, comprising:
a cap structure that selectively engages the container thereby obstructing the access opening, wherein said cap structure has an interior surface that faces into the access opening toward the consumable product; and
a plastic seal attached to said interior surface of said cap structure, wherein said plastic seal is fabricated from a composition that includes a plastic and at least one scented material dissolved within said plastic in a concentration in excess of its solubility at ambient temperature.

2. The closure system according to claim 1, wherein plastic is selected from a group including poly-vinyl chloride, Plastisol, HDPE and thermoset plastics.

3. The closure system according to claim 1, wherein said cap structure is metal.

4. The closure system according to claim 1, wherein said cap structure is fabricated from a secondary plastic that is dissimilar from said plastic seal.

5. The closure system according to claim 1, wherein said cap structure is a bottle cork.

6. A container assembly for holding a consumable product, said container assembly comprising:
a container having an access opening;
a closure for selectively obstructing said access opening, wherein a head space is defined within said container between said closure and the consumable product; and
a plastic structure exposed to said head space, wherein said plastic structure is comprised of a plastic having at least one scented material which is incorporated or dissolved therein, wherein said at least one scented material is present in a concentration in excess of its solubility at ambient temperature, thereby causing at least one scented material to separate out of solution over time.

7. The assembly according to claim 6, wherein said plastic structure is a seal disposed between said closure and said container.

8. The assembly according to claim 6, wherein said container has component parts and said plastic structure is a seal used to bind at least some of said component parts.

9. The assembly according to claim 7, wherein said seal is attached to said closure.

10. The assembly according to claim 8, wherein said closure is a bottle cork.

11. The assembly according to claim 8, wherein said closure is a plastic cap.

12. The assembly according to claim 7, wherein said seal is a tamper seal that is attached to said container.

13. A method of making part of a container or closure system, said method comprising the steps of:
providing a first plastic material;
providing at least one scented material that is soluble in said first plastic material;
providing a first plastic material in a liquid state; dissolving said at least one scented material within said first plastic material to form a plastic composition, wherein at least one scented material is added to a point of solution saturation when said first plastic material is in said molten or liquid state; and
forming a part of the container closure system from said plastic composition.

14. The method according to claim 13, further including the step of assembling said part into said container closure system.

15. The method according to claim 14, wherein said step of forming said part includes molding a closure seal from said plastic composition.

16. The method according to claim 15, wherein said step of assembling said part into said container closure includes attaching said closure seal to said container closure or container.

17. The method according to claim 16, wherein said container closure is selected from a group consisting of metal caps, plastic caps, tamper seals and corks.

18. A method of directing migration of scented materials from an aromatized plastic material, said method comprising the steps of:
providing a plastic composition containing at least one plastic material and at least one scented material that saturates said at least one plastic material; and
positioning said plastic composition against an air impervious material, wherein said at least one scented material migrates out of said at least one plastic material toward surfaces not covered by said air impervious material.

19. The method according to claim 18, wherein said air impervious material is the inside of a consumable products packaging.

20. A method of delivering a stable aroma and flavor profile to ingestibles held within a confined container, said method comprising the steps of:
adding flavored materials to at least one plastic in a concentration in excess of its saturation at ambient temperature, therein forming a plastic composition, wherein said flavored material are kept stable and unoxidized within said plastic composition until said flavored material migrate out of said at least one plastic and are exposed to air; and
positioning said plastic composition within a confined container of a consumable.
